(19) United States
(12) Patent Application Publication
(10) Pub. No.: US 2026/0278073 A1
(43) Pub. Date: Sep. 17, 2026

(54) SYSTEMS AND METHODS FOR PROTECTING DATA USING DIGITAL SCHEMAS

(71) Applicant: Click Therapeutics, Inc., New York, NY (US)

(72) Inventors: Kevin Appelbaum, Bend, OR (US); Mark Berman, Austin, TX (US); Andres Camacho, Lafayette, CA (US); Kristin Wynholds, Lafayette, CA (US); Anitra Appa, San Francisco, CA (US); Martha Simmons, Belmont, CA (US); Prapti Mehta, San Anselmo, CA (US)

(73) Assignee: Click Therapeutics, Inc., New York, NY (US)

(21) Appl. No.: 19/079,412

(22) Filed: Mar. 13, 2025

Related U.S. Application Data
(63) Continuation of application No. 18/690,246, filed on Sep. 7, 2022, now abandoned.
(60) Provisional application No. 63/241,434, filed on Sep. 7, 2021.

(51) Int. Cl. G06F 21/55; G09B 5/02

(52) U.S. Cl. CPC G06F 21/552; G09B 5/02; G06F 2221/034

(57) ABSTRACT

Various examples, systems, and/or methods are disclosed relating to employing nutritional cognitive behavioral therapy. One or more processors can provide a self-monitoring tracker feature in a mobile application to a user, perform self-monitoring of responses or biometric data, identify endpoints for the user to achieve by applying at least one artificial intelligence (AI) model, and update digital schema based on the identified endpoints.

### TECHNICAL FIELD

The present disclosure relates to systems and methods for managing digital schemas in a computational environment to protect data integrity, enforce structured behavioral interventions, and facilitate adaptive learning processes. More specifically, implementations described herein pertain to machine learning-based digital frameworks that structure and modify schema-driven interventions, track biometric and behavioral data, and dynamically adjust user-specific response models using artificial intelligence (AI) processing.

### BACKGROUND

Despite the availability of a wide range of pharmacological treatments for type 2 diabetes, it is estimated that as many as half of U.S. patients with type 2 diabetes are not achieving glycemic control. It also has been determined that these pharmacological treatments may be insufficient in many cases. Moreover, even when adequate glycemic control is achieved via pharmacotherapy, the pharmacotherapy itself can produce significant deleterious side effects, and a substantial residual risk to all-cause mortality can still remain. Importantly, pharmacological treatment also does not get at the behavioral determinants of type 2 diabetes. These determinants, which remain largely unaddressed by conventional medical treatment, are a significant contributor to both poor glycemic control and mortality risk.

Behaviors, including dietary pattern and exercise, are known to play a role in the development and progression of type 2 diabetes and other cardiometabolic conditions. However, these behavioral determinants are resistant to change because they are created and reinforced in various ways, including but not limited to personal habits, societal behavioral norms, and culturally-based ideas. Therapy that targets such behaviors to promote and facilitate diabetes care has been attempted, but there are numerous barriers to success. Among these are limitations in the health care system itself, as it is not organized to provide comprehensive behavioral therapy at the required scale and on the necessary time frame. There are different health care providers, some very large, some very small, some focusing on pharmacotherapy, some focusing on behavioral therapy, as well as individual doctors in remote locations having varied skill sets and experiences. Implementing the necessary modalities and connections to accommodate all of these variants and variabilities in the health care system is daunting. Consequently, primary healthcare providers utilizing conventional pharmacotherapies presently lack the ability to provide or prescribe effective behavioral therapy to their patients, and particularly on a daily or weekly basis.

What is needed, then, is a therapeutic intervention that can deliver effective behavioral therapy at scale, and on a weekly if not daily basis, so as to leverage and bolster the trust established in a patient-provider relationship, and to provide actionable data back to both provider and patient to advance patient care. The present invention addresses this and other unmet needs.

### SUMMARY

The present invention provides Nutritional Cognitive Behavioral Therapy (Nutritional-CBT) to treat patients with type 2 diabetes and other cardiometabolic diseases. Nutritional-CBT is an adaptation of CBT that is designed specifically to address the cognitive patterns and mental structures that drive dietary patterns and associated lifestyle behaviors, to help patients with such diseases and disorders. Nutritional-CBT builds on traditional CBT by systematically targeting the cognitive structures, behavioral routines, emotional patterns and coping skills that underlie culturally-specific eating behaviors.

Some implementations relate to a method for using machine learning, including providing, by at least one processor, a self-monitoring tracker feature in a mobile application to a user, the self-monitoring tracker including a digital schema, including a series of lessons, wherein at least one lesson addresses one or more beliefs corresponding to one or more behaviors of the user and a plurality of exercises to provide an improvement corresponding to the one or more beliefs or to initiate a new behavior. The method can include performing, by the one or more processors, self-monitoring by tracking at least one of a plurality of responses or biometric data from the user corresponding with at least one lesson of the series of lessons or at least one exercise of the plurality of exercises, identifying, based on the self-monitoring, one or more endpoints for the user to achieve during or after a lesson or an exercise by applying at least one artificial intelligence (AI) model, and wherein the at least one artificial intelligence (AI) model is updated using the plurality of responses of at least the user or biometric data from at least the user, and updating the digital schema based on one or more identified endpoints.

Some implementations relate to a method, further including dynamically adjusting the endpoints for the user between consecutive lessons or exercises, wherein the dynamic adjustment applies one or more machine learning algorithms trained using the plurality of responses and biometric data from a plurality of users.

Some implementations relate to a method, further including modifying, for the user, a subsequent one of the lessons or at least one interactive exercise using the at least one processor, wherein the modifying applies one or more machine learning algorithms.

Some implementations relate to a method, wherein the lesson is specific to a particular condition that the self-monitoring tracker is treating, or to understanding, addressing, or controlling particular human physiological attributes, or to understanding, addressing, or controlling particular human physiological responses, or developing certain desirable behaviors.

Some implementations relate to a method, further including generating one or more personalized notifications and communicating the one or more personalized notifications to the user, the one or more personalized notifications selected from the group consisting of guidance, progress reporting, treatment score, reminders, nudges, and rewards.

Some implementations relate to a method, wherein the identifying relies at least in part on performance by the user in reaching previously set endpoints.

Some implementations relate to a method, wherein one or more of the series of lessons are interactive, and the user inputs information into the self-monitoring tracker either voluntarily or as the self-monitoring tracker provides a prompt.

Some implementations relate to a method, further including providing biometric notifications via a protected data channel in response to entry of the biometric data of the user, optionally wherein the biometric notifications indicate danger levels.

Some implementations relate to a method, the method further including determining one or more treatment changes or behavioral modifications for the user.

Some implementations relate to a system for machine learning, including: one or more processors coupled with memory, configured to: provide a self-monitoring tracker feature in a mobile application to a user, the self-monitoring tracker including a digital schema, including: a series of lessons, wherein at least one lesson addresses one or more beliefs corresponding to one or more behaviors of the user; a plurality of exercises to provide an improvement corresponding to the one or more beliefs or to initiate a new behavior; perform self-monitoring by tracking at least one of a plurality of responses or biometric data from the user corresponding with at least one lesson of the series of lessons or at least one exercise of the plurality of exercises; identify, based on the self-monitoring, one or more endpoints for the user to achieve during or after a lesson or an exercise by applying at least one artificial intelligence (AI) model, and wherein the at least one artificial intelligence (AI) model is updated using the plurality of responses of at least the user or biometric data from at least the user; and update the digital schema based on one or more identified endpoints.

Some implementations relate to a system, wherein the one or more processors are further configured to dynamically adjust the endpoints for the user between consecutive lessons or exercises, wherein the dynamic adjustment applies one or more machine learning algorithms trained using the plurality of responses and biometric data from a plurality of users.

Some implementations relate to a system, wherein the one or more processors are further configured to modify, for the user, a subsequent one of the lessons or at least one interactive exercise using the at least one processor, wherein the modifying applies one or more machine learning algorithms.

Some implementations relate to a system, wherein the lesson is specific to a particular condition that the self-monitoring tracker is treating, or to understanding, addressing, or controlling particular human physiological attributes, or to understanding, addressing, or controlling particular human physiological responses, or developing certain desirable behaviors.

Some implementations relate to a system, wherein the one or more processors are further configured to generate one or more personalized notifications and communicate the one or more personalized notifications to the user, the one or more personalized notifications selected from the group consisting of guidance, progress reporting, treatment score, reminders, nudges, and rewards.

Some implementations relate to a system, wherein identifying relies at least in part on performance by the user in reaching previously set endpoints.

Some implementations relate to a system, wherein one or more of the series of lessons are interactive, and the user inputs information into the self-monitoring tracker either voluntarily or as the self-monitoring tracker provides a prompt.

Some implementations relate to a system, wherein the one or more processors are further configured to provide biometric notifications via a protected data channel in response to entry of the biometric data of the user, optionally wherein the biometric notifications indicate danger levels.

Some implementations relate to a system, wherein the one or more processors are further configured to determine one or more treatment changes or behavioral modifications for the user.

Some implementations relate to a non-transitory computer readable medium (CRM) including one or more instructions stored thereon and executable by one or more processors to: provide a self-monitoring tracker feature in a mobile application to a user, the self-monitoring tracker including a digital schema, including: a series of lessons, wherein at least one lesson addresses one or more beliefs corresponding to one or more behaviors of the user; a plurality of exercises to provide an improvement corresponding to the one or more beliefs or to initiate a new behavior; perform self-monitoring by tracking at least one of a plurality of responses or biometric data from the user corresponding with at least one lesson of the series of lessons or at least one exercise of the plurality of exercises; identify, based on the self-monitoring, one or more endpoints for the user to achieve during or after a lesson or an exercise by applying at least one artificial intelligence (AI) model, and wherein the at least one artificial intelligence (AI) model is updated using the plurality of responses of at least the user or biometric data from at least the user; and update the digital schema based on one or more identified endpoints.

Some implementations relate to a CRM, wherein the one or more instructions stored thereon and executable by the one or more processors are further configured to: dynamically adjust the endpoints for the user between consecutive lessons or exercises, wherein the dynamic adjustment applies one or more machine learning algorithms trained using the plurality of responses and biometric data from a plurality of users.

### BRIEF DESCRIPTION OF THE DRAWINGS

Aspects of the present invention now will be described in detail with reference to the accompanying drawings, in which:

FIG. 1 is a diagram providing an overview of aspects of embodiments;

FIG. 2 is a bar chart showing results of varying degrees of use of nutritional CBT and corresponding FBG results;

FIG. 3 is a bar chart showing results of varying degrees of presence of plant-based nutrition in patient diets, and corresponding FBG results;

FIG. 4 is a bar chart showing results of varying degrees of exercise and corresponding FBG results;

FIG. 5 is a bar chart showing results of varying degrees of use of nutritional CBT and corresponding weight loss results;

FIG. 6 is a bar chart showing patient feedback regarding a digital therapeutic applying nutritional CBT without benefit of Al/ML;

FIG. 7 is a high-level block diagram depicting aspects of a nutritional CBT system according to an embodiment;

FIG. 8 is a high-level block diagram of one of the elements of a nutritional CBT system according to an embodiment.

FIG. 9 is a graph showing a greater reduction in Ale for patients on a nutritional CBT system as compared to control group;

FIG. 10 is a bar chart showing gradual and steady improvements in fasting blood glucose levels, for patients on a nutritional CBT system as disclosed herein;

FIG. 11 is a graph showing trends in fasting blood glucose level for three different therapies (a nCBT system as herein disclosed, GLP1, and SGLT2);

FIG. 12 is a bar chart showing that Cardiovascular Outcome Trials (CVOTs) show lower relative Ale reduction compared with new drug pivotal for same drug;

FIG. 13 is a bar chart showing that higher dose of nCBT lessons completed is associated with larger Ale improvements at 180 days;

FIG. 14 is a graph showing that a higher nCBT dose subgroup shows substantially greater Ale improvement compared to standard of care (SOC) control group;

FIG. 15 is a bar chart illustrating significant improvements in Ale levels in patients on a nCBT system as described herein, despite use of fewer diabetes medications;

FIG. 16 is bar chart showing that patients on a nCBT system as herein disclosed show a range of large improvements in Ale levels at 180 days;

FIG. 17 is a bar chart showing that higher nCBT dose as herein described is associated with larger improvements, but not higher rates of adverse events (AEs)

FIG. 18 is a bar chart illustrating that antihyperglycemic medication utilization and healthcare utilization increased more in the SOC control group patients than those on an nCBT system as herein disclosed, over 6 months.

### DETAILED DESCRIPTION

Physicians and other clinicians, as well as scientists and other scientifically-trained professionals, often share data or results of their work, in the form of journal articles, conference reports, and the like. Such dissemination is intended to advance scientific and/or medical knowledge and learning, and enable application of that imparted information to further patient treatment, for example.

There are limits to the extent and amount of information dissemination and consequent improvement in clinical results, however. The practicality of individual physicians and other clinicians, whether in a large teaching hospital or in smaller, more remote locations, to actually take advantage of the disseminated information is limited because there is only so much information that an individual doctor or clinician, or even an assembled team of doctors and/or clinicians, can assimilate and apply, or in the case of individual physicians, even obtain. In addition, different sources may provide supplements or other augments to existing information, requiring individuals or even teams to engage in frequent “refreshing” of knowledge and consequent learning of effects on treatment regiments.

Aspects of the present invention provide practical application to computer technology, to expedite provision of patient treatment, and to improve patient outcomes, in ways that prior sharing of information cannot. The content and delivery mechanisms of nutritional-CBT in accordance with aspects of the present invention leverage experience and data from clinician-patient and health coach-patient interactions among substantial patient populations to distill common maladaptive thinking and beliefs pertaining to diet and lifestyle.

In an embodiment, a digitally-delivered therapy can be widely disseminated to large patient populations, yet personalized to the individual patient using artificial intelligence (AI)/machine learning (ML) driven feedback loops. The subject treatment plans provide patient lessons, skill exercises and goals based on a wide range of data from substantial numbers of patients, reflecting many different combinations of physiological, biometric, and psychological characteristics of those patients and corresponding treatment results, yield far more informed and effective treatments because individual physicians or clinicians, even in large hospitals, are unable to assimilate the data the way an Al/ML system can.

According to aspects of the present invention, digitally-delivered nutritional-CBT involves, among other things, one or more of the following: Identifying and measuring maladaptive thoughts based on misinformed or false underlying core beliefs (e.g., those related to macronutrient fears, the hedonic nature of eating, physical exertion, other perceived barriers to changing lifestyle) that lead to disease-promoting behaviors; Replacing these maladaptive core beliefs and thought patterns with adaptive ways of thinking developed from rational reflection; Providing collaborative (between patient and digital therapeutic) construction of behavioral exercises to test core beliefs and set goals for improvement; Using additional validated behavioral techniques to enhance a patient's capacity to solve problems, plan behaviors, and cope with interfering emotions or thoughts.

A digital therapeutic according to an embodiment delivers treatment to patients with type 2 diabetes to target behaviors related to achieving glycemic control so as to reduce HbA1c. In an embodiment, the digital therapeutic may be downloaded to a patient's smartphone to deliver nutritional-CBT.

Throughout the description of embodiments of the present invention, terms such as “participant,” “patient,” and “subject” may appear. These terms are used interchangeably throughout.

In an embodiment, the digital therapeutic may ask patients to answer behavioral intake questions, as a behavioral assessment. Questions may focus on current and recent biometric data, eating and drinking habits, exercise habits, and the like. Things like family history, and current family, living, and work situations also may be relevant. Additionally or alternatively, some of the topics in the behavioral intake questions may be pursued in more detail in one or more of the therapy lessons, and/or may be a focus of one or more skill-based exercises that may be associated with the various therapy lessons.

During the behavioral assessment, and/or subsequently during treatment, patients may be asked to assess the presence and strength of their beliefs and perceived barriers to achieving diet and exercise patterns that are sufficient to improve glycemic control. A goal of this behavioral assessment is to help patients identify their unconscious beliefs that may be responsible for poor behavioral habits or may represent barriers to adopting new helpful habits that influence glycemic control. Different patients will respond differently to questions asked. Variations can depend on numerous patient characteristics, including but not limited to age, height, weight, health symptoms, living and eating habits, geography, culture, race, and other demographic, physiological, and psychological characteristics. Aspects of the invention use patient responses to tailor the treatment presented, taking advantage of Al/ML learning from vast quantities of other patient data and responses by analyzing and interpreting data in a way that individual physicians and clinicians are incapable of doing.

Al and ML help to reveal the right treatment pace, intensity and support needed to maximize efficacy for each individual. Examples of Al/ML techniques and algorithms that usefully may be employed include, but are not limited to, Linear Regression, Logistic Regression, Linear Discriminant Analysis, Classification and Regression Trees, Decision Trees, Clustering, Inductive Logic Processing, Stochastic Modeling, Statistical Modeling, Case-Based Reasoning, Bayes and Naive Bayes, K-Nearest Neighbors (KNN), Learning Vector Quantization (LVQ), Support Vector Machines (SVM), Random Forest, Boosting, and Ada Boost. Ordinarily skilled artisans also will appreciate that certain types of machine learning algorithms, such as convolutional neural networks (CNN) and recurrent neural networks (RNN) (sometimes referred to as long short term memory (LSTM), which employ backpropagation, as well as recurrent convolutional neural networks (RCN N) and other types of artificial neural networks, may be particularly suitable.

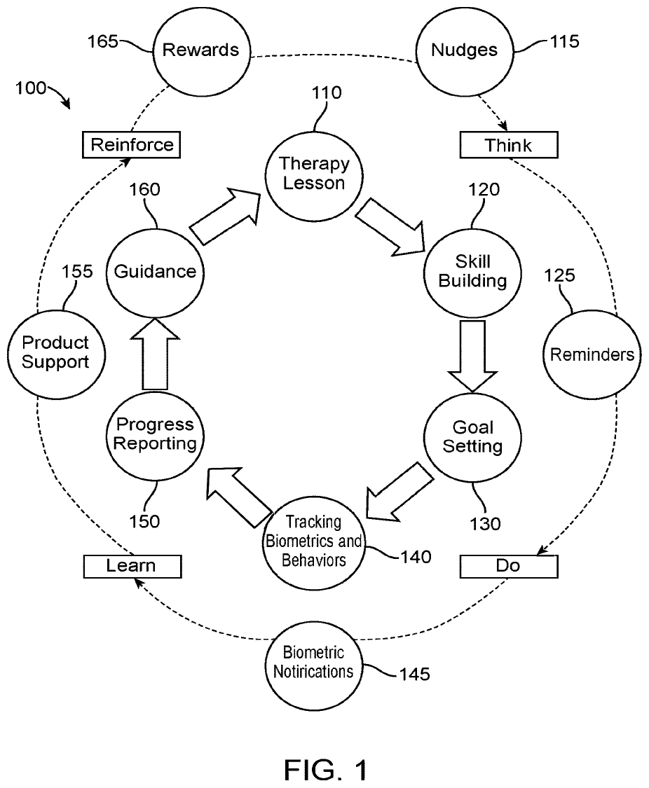

FIG. 1 is a high level diagram providing an overview of what various aspects of embodiments of the invention accomplish. The diagram appears in a somewhat circular form, with a “therapy lesson” bubble at the top. A given therapy lesson can begin a cycle of therapy and subject activity, leading to the next therapy lesson. Various ones of the elements summarized below also will be discussed in more detail herein.

Each therapy lesson 100 can help to isolate and to shift a specific set of beliefs that are barriers to change. Each lesson 100 is or can be interactive. CBT techniques help to identify and shift false beliefs and ideas in a non-threatening, non-judgmental manner. The subject gradually advances from early concepts in early therapy lessons, to allow time for cognitive restructuring before addressing more deeply held beliefs. The therapy creates emotional resilience needed to make enduring changes.

As part of the therapy lesson, the digital therapeutic may provide one or more personalized notifications including, e.g., guidance, progress reporting, treatment scores, reminders, nudges, and rewards. These can, among other things, prompt subjects to think more closely about the lesson; direct subjects to expand their repertoire of activities, perhaps discovering previously undiscovered skills; and even encourage subjects who already are on track to exceed their goals, and/or set higher goals.

These personalized notifications can also give encouragement and suggest activities to reinforce behaviors, as well as help subjects celebrate progress and provide guidance as to what to do next to practice healthy behaviors. In some embodiments, reinforcement and/or celebration is provided when a goal is achieved. In other embodiments, guidance and/or additional actions to take are provided when goals have not been achieved.

Each therapy lesson is followed by skill building 120. Skill-building modules can reinforce ideas introduced In therapy lessons and put them into practice, initiating new behaviors. In an embodiment, in and among these skill building modules, subjects may receive reminders about desired activity and/or meal planning. Reminders 125 also can prompt subjects to check in and report biometrics and behaviors, promoting interaction with the digital therapeutic.

As a subject goes through a therapy lesson and works on skill-building, in an embodiment algorithmically prescribed weekly goals as part of goal setting 120 personalize therapy and encourage biometric self-monitoring. A treatment algorithm dynamically adjusts goals to maximize treatment response, and provides various personalized feedback loops that help sustain engagement. The subject is able to reflect on accomplishments and activities of the previous week, and review and commit to prescribed goals. In an embodiment, the subject may receive the algorithmically prescribed goals, and alter them, or pick different goals entirely. Interacting with the digital therapeutic can provide guidance to the subject to select achievable goals that are sufficiently ambitious to enable continued progress, but not so ambitious as to be discouraging.

Regular self-monitoring of behaviors and biometrics through tracking 140 can enhance self-efficacy and safety. Providing data to the digital therapeutic in the form of biometric data and behavioral data (in some cases, akin to progress reporting, mentioned below) enables the digital therapeutic to react appropriately to help the subject to continue to progress. In an embodiment, algorithmically calculated biometric notifications or alerts 145 may be triggered when dangerous values or patterns are detected. For example, FBG or blood pressure may be too high or too low. Non¬≠ dismissible information from the digital therapeutic can provide more context around the subject's situation and also can provide next steps to follow.

To help subjects remain on track, progress reporting 150 can connect changes in behavior to measurable health improvements week over week. In an embodiment, progress reports 150 can include a treatment. Progress reports 150 can show how a subject is doing in relation to each of the biometrics and behaviors they are tracking. Subjects are able to learn about their progress, and about how to make better progress, from the progress reports and treatment score.

In an embodiment, subjects may contact Product Support 155 from within the digital therapeutic to help them resolve technical or usability issues with the digital therapeutic, and keep the subjects interested and engaged, avoiding discouragement and/or frustration.

In an embodiment, the digital therapeutic can provide guidance 160 to address common behavioral barriers and misperceptions. When users are off-track or stuck in their progress, guidance 160 can provide additional insights and information. Interactive and visual content can deliver information and insights in a digestible and actionable way.

In embodiments, a subject can receive reinforcement and rewards 165 for various accomplishments. For example, the subject may have consecutive streaks of exercise days or selection of plant-based meals, or may have achieved a drop in FBG or blood pressure or weight. The digital therapeutic can provide rewards to the subject, which in an embodiment may be displayed as trophies in a virtual trophy case. Subjects can review their treatment journey, and can access content, such as journals, voice recordings, and photographs, that they have created during treatment. The rewards and the trophy case display can reinforce and encourage subjects, as well as promote repetition of skills for mastery.

The cycle that FIG. 1 depicts can be repeated as a subject progresses through each lesson. In an embodiment, Al/ML algorithms may determine that a lesson needs to be repeated (if a subject has not shown enough progress during the time between lessons), or that a lesson may be skipped (if a subject has shown enough progress, and/or mastery of a skill to be worked on in the course of a particular lesson), or that lessons should be reordered to optimize the subject's progress.

The subject can experience all of these steps as activities that stimulate conscious thought about what drives their behaviors. Subjects can be introduced to new ideas, and assisted in putting those new ideas into practice. Subject can learn from experience, making them receptive to guidance on how to make behavioral changes. What results is a dynamic adjustment of a subject's maladaptive beliefs.

In an embodiment, the digital therapeutic helps patients understand the steps they should prioritize by presenting them with a treatment plan that summarizes their daily and weekly goals. Each week, the digital therapeutic may ask patients to complete a new nutritional-CBT module, along with one or more skill-based exercises that are related to that particular week's nutritional-CBT module. All patients potentially have access to the same course of nutritional-CBT modules, but as noted above, in an embodiment the course is tailored for patients based on volumes of other patient data and responses. In an embodiment, the nutritional-CBT modules may take between 10-20 minutes to complete. As noted earlier, nutritional-CBT modules may be referred to as “therapy lessons” within the digital therapeutic.

In an embodiment, each therapy lesson may address core patient beliefs in one or more of the following areas:
Personal beliefs and barriers, such as those related to a patient's ability to change and control his or her behaviors;
Beliefs about macronutrients and the importance of various food types;
Hedonic-related beliefs about pleasant or unpleasant sensations experienced by eating or exercising;
Beliefs about exercise;
Beliefs about sleep, stress and social interaction.

One purpose of these therapy lessons is to bring forth unconscious beliefs, automatic thoughts (the spontaneously occurring verbal or imaginary mental activity that occurs involuntarily in response to a situation), emotions, and attentional bias (the inappropriate focus of attention on specific information over others that distorts perception) into patients' conscious awareness. Additionally, the therapy lessons and associated skill-based exercises can empower patients to challenge these now conscious thoughts and beliefs by recommending more helpful ideas and behavioral practices. These lessons and exercises allow patients to begin correcting false beliefs and replacing them with consciously adopted thoughts and beliefs that drive disease-reversing behaviors.

In an embodiment, each therapy lesson may comprise one or more of the following core steps:
Identifying false or unhelpful beliefs;
Selecting alternative, more helpful beliefs;
Planning one or more related skills to practice.

The first step helps patients to recognize specific false beliefs and to illustrate how these beliefs may promote behaviors that can worsen type 2 diabetes or other cardiometabolic disorders. To help the patient identify and assess specific false beliefs, in an embodiment the system may present, via the digital therapeutic: multiple-choice quiz questions; patient vignettes; and/or contrasting views of false and alternative beliefs.

The second step helps to expose patients to adaptive alternatives to supersede unhelpful thoughts and beliefs. In an embodiment, the system may present a patient with one alternative belief statement at-a-time and may ask the patient to rate how strongly they currently believe the statement to be true for them. The system then may instruct the patient to vocalize the belief statement with which they identify most strongly, and to record (for example, in a video, audio, and/or text-based journal) about a relevant experience. This rating and journaling process can help to reinforce the idea that beliefs and thoughts can be changed, and that consciously choosing to repeat a new adaptive thought or idea is a key step in the process.

The third step helps patients to plan one or more related skills to practice, so as further to reinforce the alternative thoughts learned in a particular therapy lesson. In an embodiment, the system may ask patients to browse up to five skill options and to select one or more options to practice during the week.

As with face-to-face therapy, the patient is prompted to engage with each of these three steps by considering their unique beliefs, ideas, and life experiences. Each lesson also offers a small number of options for completing each step to allow the patient to further personalize the therapeutic process. Patients can also repeat lessons and skills as needed and gain access to future lessons via a lesson library. In an embodiment, the digital therapeutic analyzes patient-generated data from use of the digital therapeutic to automatically provide feedback to the patient about their degree of engagement in the nutritional-CBT process. In an embodiment, the feedback is analyzed in the context of vast quantities of other patient responses about degree of engagement, taking into account patient personal, cultural, and/or health characteristics.

In an embodiment, also as noted earlier, skill exercises may be provided to improve patients' dietary, exercise, or supportive behavioral patterns. Patients' practice of these skills can enhance executive function tasks such as planning, problem-solving, and goal setting. In an embodiment, each therapy lesson explains the rationale and benefits of the skill exercise or exercises in the lesson, in the context of the core belief topic being explored. In an embodiment, lesson topics may include some or all of the following:

TABLE 1

| Lesson | Title |
|---|---|
| 0 | Exploring our Beliefs: Explore the concept that false beliefs |
| | can underlie unhealthy behavior |
| 1 | Ideas about Health/Type 2 Diabetes: Examine ideas that |
| | prevent people from using lifestyle change to improve their |
| | health, including type 2 diabetes or other cardiometabolic |
| | diseases |
| 2 | Blood Sugar/Carbohydrates: Examine ideas about |
| | carbohydrates and blood sugar that prevent people with |
| | diabetes from eating healthful whole plant foods |
| 3 | Protein: Examine ideas about protein which prevent people |
| | from replacing meat, dairy, and eggs with whole plant foods |
| 4 | Affordability: Address the misconception that eating whole |
| | plant foods must be expensive |
| 5 | Exercise: Address false ideas about exercise which prevent |
| | people from increasing their weekly exercise minutes |
| 6 | Hunger: Address false beliefs about what hunger indicates |
| 7 | Weight: Explore ideas about the impact of moderate weight |
| | loss on type 2 diabetes |
| 8 | Comfort Food: Explore ideas about the emotions that can |
| | lead to eating unhealthy comfort foods |
| 9 | Control: Explore the ideas about the relative values of |
| | skillpower and willpower |
| 10 | Loyalty: Address unhelpful ideas about traditions that can |
| | prevent successful lifestyle changes during social functions |
| 11 | Our Ability to Change: Address ideas about mindset and |
| | individual ability to change behavior |
| 12 | Healing: Explore ideas about setbacks during successful life |
| | changes |
| 13 | Power of Beliefs: Explore the power of ideas and how |
| | mindsets shift during the program |
| 14 | Stress: Explore the idea that everybody experiences the |
| | range of different kinds of stress |
| 15 | Response to Stress: Explores ideas about developing skills |
| | and curating resources to respond to stress in healthy ways |
| 16 | Sleep: Addresses ideas about restorative sleep and its |
| | importance for health |
| 17 | Connection: Explore ideas about authentic connections to |
| | others as a key component of good health |
| 18 | Opportunity: Explore ideas about craving resulting from |
| | past experiences rather than physiologic need |
| 19 | Meaning: Explore the idea that people can create |
| | opportunity for themselves even under difficult |
| | circumstances |
| 20 | Purpose: Explore ideas about the possibility of meaning in |
| | everything, even in difficult situations |
| 21 | Strength/Resistance Exercise: Address ideas about having |
| | reasons and purpose for changes beyond personal health |
| 22 | Caring for Ourselves: Explore ideas around building muscle |
| | strength and its importance in health |
| 23 | Empowerment: Explore the idea that caring for ourselves is |
| | fundamentally an attitude, rather than specific actions |
| 24 | Craving: Address the idea of empowerment in terms of |
| | transition from external to internal motivation |
| 25 | Our Evolving Selves: Explore ideas around expected shifts in |
| | self-identity that come with personal growth |

As ordinary skilled artisans will appreciate from the foregoing list, some of the lessons may be specific to the particular condition that the digital therapeutic is treating (for example, type 2 diabetes or another cardiometabolic disorder), while others may be more broadly applicable to cardiometabolic disorders in general. For example, some of the lessons may be specific to understanding, addressing, and/or controlling particular human physiological attributes (e.g, blood sugar, blood pressure, heartbeat, weight). Some of the lessons may be specific to understanding, addressing, and/or controlling particular human physiological responses (e.g., hunger, thirst, craving). Some of the lessons may be specific to developing certain desirable behaviors (e.g., stress reduction, exercise, rest, sleep).

Remarkably then, the digital therapeutic of the present invention is readily adaptable to the treatment of a broad set of cardiometabolic diseases beyond type 2 diabetes, including gestational diabetes, hypertension, obesity, dyslipidemia, hyperlipidemia, hypertriglyceridemia, non-alcoholic fatty liver disease, non-alcoholic steatohepatitis, hypercholesterolemia and familial hypercholesterolemia, heart disease, coronary artery disease, and chronic kidney disease, and with only minimal changes to most of the lesson plans and/or skill exercises. The subject digital therapeutic thereby leverages and scales the power of targeting and changing core maladaptive beliefs and behaviors underlying a wide range of cardiometabolic diseases.

In an embodiment, a patient may follow a lesson plan such as the one outlined above. In an embodiment, a plurality of patients may start out with the same lesson plan. In an embodiment, different patients may be presented with different lesson plans. Among the criteria which may yield different lesson plans are patient age, height, weight, body mass index, and pre-existing conditions, if any.

Along the way, different patients, making different amounts of progress, may have their lesson plans changed. The lesson plan may be tailored to the patient later on, even if the initial lesson plan starts out the same as for other patients. In an embodiment, the lesson regimen may take a predetermined amount of time, for example, 90 days. There may be a plurality of different lessons, for example, 26. In an embodiment, the lessons may be split into two groups. Depending on a patient's progress or reaction to particular lessons, one or more lessons may be repeated one or more times. In an embodiment, depending for example on a patient's background or progress through the CBT regimen, one or more lessons may be skipped.

In an embodiment, a practical application of this digitally-based therapy may reside in the ability to draw on a substantial body of patient experience and physiology, involving some or all of the criteria listed earlier. In addition, other aspects of patient history and experience, including prior efforts at controlling or adjusting living, sleeping, and dieting habits, may dictate the tailoring of a lesson plan to a patient.

In an embodiment, lessons may be delivered on a weekly basis. For example, the 26 lessons mentioned above, may be provided over 180 days, approximately one lesson per week. In an embodiment, the lesson selection and sequence for each patient may be the same. The experience with one or more of the lessons may be interactive, wherein the subject may either be prompted for or may volunteer information relevant to the part of the lesson being covered, and may input that information into the digital therapeutic. In an embodiment, a patient's progress through the lessons may be determined by an amount or degree to which the patient may interact during a given lesson. More interaction may promote faster progress from one lesson to another, or skipping of one or more lessons. Less interaction may result in slower progress from one lesson to another, or in repeating of a lesson with less interaction.

In an embodiment, lessons may be accessed on a handheld device such as a phone or tablet, or on a desktop or laptop computer. Different kinds of interactive media may be employed. For example, some lessons may be mainly text. Some lessons may involve audio and/or video recordings. Some or all of the lessons may involve quiz, type questions, to help to ascertain a patient's progress through a particular lesson, or a patient's retention of information imparted during a particular lesson.

In an embodiment, some lessons may include exercises for a patient to perform interactively. For example, a patient may be asked to record information in a journal, either by text or by audio or video recording. The types of information may be physiological in nature (for example, weight, food intake, type and/or amount of exercise, or the like), or may be more personal in nature (for example, reaction to particular types of changes in diet and/or exercise).

In an embodiment, a patient's journaling may be even more personal in nature. For example, a patient may be asked to record feelings about particular lifestyle-related statements. A statement to which a patient somehow expresses a particular level of agreement or affinity may be presented to the patient as an aspect of behavior to adopt or implement.

In an embodiment, one of the exercises may require the patient to select a particular skill module from among a plurality skill modules. That skill module then may become something on which the patient works during the coming week or other time interval between lessons. In an embodiment, the skill module may be an interactive exercise, in which the patient may respond to questions, or may provide evidence of performing the particular skill (for example, posting a picture of the patient performing the skill).

In an embodiment, each lesson may be associated with one or more skills. In an embodiment, as many as five skills may be involved in a particular lesson, and as many as 96 skills over the entire 180 day discipline, but the specific numbers are not critical. In going through a lesson, a patient may select a particular skill on which to work, and may follow through on that work in self-directed fashion throughout the lesson.

In an embodiment, a patient may be asked to set performance goals for a coming period of time, for example, a week, a bi-week, or a month. The performance goals may include specific meal disciplines, for example, type of food, amount, when consumed, and the like. In an embodiment, the performance goals may include exercise minutes, which may be for a particular type of exercise, or may be divided among different types of exercise. Some exercise may be directed toward building cardiac and/or pulmonary endurance. Some exercise may be directed toward building strength and/or flexibility. Some exercise may be directed toward weight loss.

In an embodiment, one or more of the machine learning algorithms in accordance with aspects of the invention may recommend one or more performance goals, based on data relating to similarly-situated patients, including not only patient statistics, but also how often and/or how well similarly-situated patients have met the goals. Goals may relate to meals (frequency, intake amounts, intake types, and the like) and/or exercise minutes, which may include different types of exercise of different intensities for different periods of time, either alone or in combination.

For example, a patient may have poor eating and exercise habits. Part of the overall objective is to improve the patient's eating and exercise habits. Performance goals may be set, or the patient may set the goals themselves, to achieve improvement in diet and exercise. A patient may set fairly low goals. The system may accept the patient's selection without responding, or the system may suggest different, perhaps more aggressive goals in one or both areas, depending on the experience that the machine learning algorithms have accumulated and analyzed for identically, similarly, or comparably situated patients. That experience may relate to goals that such patients have set for themselves, and/or on the goals that such patients actually have been able to achieve. In an embodiment, the system may simply question the patient about their selections, to confirm that the patient is satisfied with the selection, or to allow the patient to choose more aggressive goals. In an embodiment, the system might encourage the patient to choose more aggressive goals, or may simply suggest to the patient that they might choose more aggressive goals. The system's reaction to the patient's selection also may be based, in whole or in part, on the patient's prior selections, the system having taken note of the degree of aggressiveness of the goals that the patient set previously, and the patient's ability to meet those goals.

A patient also may set fairly high or aggressive goals. The system may accept the patient's selection without responding, or the system may suggest different, perhaps less aggressive goals in one or both areas, again depending on the experience that the machine learning algorithms have accumulated and analyzed for identically, similarly, or comparably situated patients. That experience may relate to goals that such patients have set for themselves, and/or on the goals that such patients actually have been able to achieve. In an embodiment, the system may simply question the patient about their selections, to confirm that the patient is satisfied with the selection, or to allow the patient to choose less aggressive goals. In an embodiment, the system might encourage the patient to choose less aggressive goals, or may simply suggest to the patient that they might choose less aggressive goals. The system's reaction to the patient's selection also may be based, in whole or in part, on the patient's prior selections, the system having taken note of the degree of aggressiveness of the goals that the patient set previously, and the patient's ability to meet those goals.

In an embodiment, the lessons presented to a patient as part of an initial algorithm may be an ordered list, and may be the same for all patients. Ordinarily skilled artisans will appreciate that patients may be more likely to fit one profile than another, based on various physiological, personal, cultural, and economic profiles, as well as on such things as geography, access to health care, and other such characteristics as will occur to ordinarily skilled artisans. As the system is able to access more information about patients in all of these categories and more, the system may adapt its approach to intake of new patients, and the initial lesson plan and goal-setting. In an embodiment, the system also may adapt its treatment of existing patients, to the point of moving patients along at different rates depending on patient profile and/or patient performance. As the system is in use to an increasing extent, there may be data generated, for example, to tie performance to treatment effect. It may be possible to implement ever more sophisticated algorithms as a result.

In an embodiment, patients may proceed through the lessons at different rates depending on their progress, and/or on patient perception of how fast they can progress through lessons, how rapidly they can make changes in their diets and other living habits, how quickly they think they can achieve desired goals, and the like.

In an embodiment, the system can may observe how different patients interact with the system. Responsive to those observations, the system may offer different mechanisms to recommend different lesson plans or sequences, or different skills to patients.

In an embodiment, the digital therapeutic provides each patient with a treatment score, which represents real time or near real time feedback about the likelihood of patient success in treatment. In an embodiment, the treatment score is provided using Al/ML techniques, using ground truth data from a large number of patients with many different physical, physiological, biometric, and/or psychological characteristics.

In an embodiment, the digital therapeutic may provide a progress report, showing, for example, changes in biometrics relative to baseline, on a periodic basis. In an embodiment, the progress report is provided on a weekly basis, but other periodicities may be selected as necessary or appropriate. In one aspect, the algorithm may display different content, depending on one or more of the following categories:
Patient is improving and has exceeded targets.

Patient is improving but has not exceeded targets. In an embodiment, the algorithm may suggest that the patient consult additional content, or focus on prior content to help the patient conquer whatever barriers the patient appears to be facing in meeting the particular target or targets. In one aspect, the algorithm may present the patient with additional, perhaps more advanced behavioral strategies.

Patient has not improved, or shows only a little improvement. In an embodiment, the algorithm may give the patient additional guidance, and/or provide encouragement. The algorithm may determine that a less gentle, more direct or harsh approach is warranted. As more data is compiled, there will be room for even more granular messaging.

In an embodiment, the algorithm can vary the treatment plan based on one of the foregoing categories. Where necessary or appropriate, treatment can be augmented to reinforce particular aspects of behavioral therapy pertinent to the particular cardiometabolic disorder, or the patient can be directed toward implementation of more advanced behavioral strategies that were not part of the initial treatment regimen

In one aspect, the more advanced behavior strategies may be augmentations of the initial treatment regimen. In an embodiment, the augmentations may be personalized to various physiological and/or psychological aspects of the patient, as may other aspects of the treatment regimen. In one aspect, the algorithm's decisions on augmentation may be based just on patient biometrics, working also from the mass of data on similarly situated patients as discussed earlier. In an embodiment, augmentations and other treatment directions may be provided in response to an individual patient treatment score which represents a holistic view of patient health and behavior in response to the regimen. For example, if a patient is not providing sufficiently detailed or frequent reports on different behavioral aspects (for example, meal habits), subsequent treatment could focus on improvement in patient tracking and recording of such data.

In one aspect, it may be possible for the algorithm to select from a plurality of advanced strategies, based on Al/ML feedback from patients with identical or similar relevant profiles.

In an embodiment, possible Al/ML implementations may involve taking advantage of the ability to integrate data from many different sources, for many different patients, to provide the kind of individualized patient guidance that can optimize treatment and promote progress practically, in a way that individual physicians or clinicians cannot. For example, looking at treatment scores of similarly situated patients may make it possible to populate a treatment plan for a particular patient automatically, listing recommendations for actions that would be more likely to provide the best score improvements.

In an embodiment, within the same or different Al/ML algorithms, two or more feedback mechanisms may be employed, either in parallel or in unison, to enable use of treatment scores to provide progress reports and/or other feedback in more granular fashion. For example, a patient might be shown what action(s) or technique(s) seem to be working, and what action(s) or technique(s) might be more likely to improve the patient's biometric data.

In an embodiment, content-directing assessment tools may be employed to take advantage of Al/ML-based feedback. In one aspect, a patient may be asked to respond to survey questions. The patient's answers can be used to prioritize certain lessons or articles, or to tailor feedback, or to direct the patient toward different or more intense training to acquire certain skills.

In an embodiment, as alluded to earlier, particularly robust data sets from patients with a wide variety of physiological and psychological characteristics may enable the Al/ML algorithm to provide predictive analytics.

As an example of the foregoing, the Al/ML system may determine that certain psychological or behavioral characteristics should be given greater weight in determination of or modifications to lesson plans, independent of a patient's particular physiological or biometric characteristics. Alternatively, the Al/ML system may determine that certain physiological or biometric characteristics should be given greater weight in determination of or modifications to lesson plans, independent of a patient's particular psychological or behavioral characteristics.

### EXAMPLES

### Example 1: Behavioral Therapy Through Software-Based Digital Therapeutic

As part of a preliminary investigation into the utility of such an Al/ML implementation, a study was conducted to provide behavioral therapy through a software-based digital therapeutic paired with human support. In the following example, Al/ML techniques were not used. The results of the study are a precursor of the results to be achieved with Al/ML implementations according to aspects of the invention.

Human support has consisted of remote health coaching delivered telephonically and care escalation to nurses and physicians, as needed. Program effectiveness was studied to improve glycemic control in a non-blinded, single-arm interventional study in 97 adults with type 2 diabetes. After three months, a mean improvement in hemoglobin Ale (HbA1c) of 1% (SD 1.4) was observed in participants with baseline HbA1c>7%. A full description of this study can be found in a peer reviewed article, Berman et al., “Change in Glycemic Control with Use of a Digital Therapeutic in Adults with Type 2 Diabetes: Cohort Study,” JMIR Diabetes 2018:3(1):e4, incorporated by reference herein.

Knowledge gained from the use of the digital therapeutic with human support was translated into a software-only digital therapeutic configuration delivering nutritional-CBT. A going-in assumption was that this software-only configuration would improve glycemic control, but to a lower degree than a human+ software configuration.

To determine whether nutritional-CBT could improve glycemic control in patients with type 2 diabetes, data from participants who used a software-only version of the digital therapeutic was examined. Participants were recruited online for usability testing and to provide feedback on content through online surveys. Interested individuals self¬≠ identified as having a diagnosis of type 2 diabetes according to any duration, a blood glucose meter at home, and a smartphone. The analysis included participants with a baseline 3-day average fasting blood glucose (FBG)>152 mg/dL; a value corresponding to an hemoglobin HbA1c>7%. 74 adult participants were identified for inclusion. Table 2 below shows baseline characteristics of the participants.

TABLE 2

| Number of participants | 74 | |
|---|---|---|
| Age, years, mean (SD) | 55.2 | (7.0) |
| Body Mass Index, kg/m², mean (SD) | 34.7 | (7.9) |
| Glycemic medications reported, count, mean (SD) | 2.2 | (0.9) |
| Fasting blood glucose, 3-day average, mg/dL, mean (SD) | 199.4 | (45.1) |
| Estimated HbA1c, %, mean (SD) | 8.7% | (2.0) |
| Time since diabetes diagnosis, years, mean (SD) | 10.6 | (7.1) |
| Gender, % female (n) | 72% | (53) |

| | |
|---|---|
| Geographic location, U.S. states represented, count | 32 |

As part of typical use over 90 days, participants were encouraged to self-monitor their fasting blood glucose daily with a home glucometer and to enter those values directly into the app. In addition to interactive nutritional-CBT content, weekly goal, setting was used to guide participants in replacing highly-processed foods with whole foods, and steadily increase the proportion of meals coming mostly from plants. In the digital therapeutic, this was referred to as eating more “plant-based meals”.

Changes in self-reported FBG were examined by looking at the difference between 3-day averages anchored by the first and last values reported. To examine whether participants who were not tracking FBG might negatively impact population outcomes, an intent-to-treat (ITT) analysis was performed by assuming no change in FBG for those who did not track after reporting a baseline.

Mean change in FBG was −22.9 mg/di (SD 42.0) over an average of 69 days (SD 30.0), which corresponds to an estimated HbA1c change of −1%. Table 3 below shows changes in FBG.

TABLE 3

| | | Fasting Blood Glucose, mean (SD) | | |
|---|---|---|---|---|
| Participant | | Baseline | Last | |
| Sub-group | n | Average | Average | Change |
| All | 74 | 199.4 (45.1) | 176.5 (49.8) | −22.9 (42.0) * |
| Stable glycemic | 65 | 197.7 (43.8) | 176.0 (49.2) | −21.7 (40.2) * |
| Baseline | 65 | 187.4 (32.6) | 168.4 (45.1) | −19.0 (40.1) * |
| HbA1c 7-11% | | | | |

* Paired t-test comparing baseline average to most recent average, p < 0.001

In the study, a non-Al/ML version of the digital therapeutic was used in a real-world setting while participants continued usual medical care. Participants were free to change medications as directed by their physician and were encouraged to adhere to prescribed medications and report any changes within the app. The impact of changes to glycemic medications while using the app was examined. At baseline, 93% (69/74) of participants reported using anti-hyperglycemic medications. Over the 90—days observed, nine of the 69 participants reported a change (dose or count of meds); two reported a decrease and seven reported an increase. Exclusion of these nine participants from the analysis did not have a meaningful effect on the mean improvements in FBG observed.

It was considered desirable to examine whether outliers might overly influence outcomes and to align with the inclusion criteria for an upcoming randomized controlled trial. Accordingly, the mean change in FBG in participants with an estimated baseline HbA1c between 7 and 11% was examined. In these 65 participants, the mean change in FBG was −19.0 mg/dL over a mean of 71 days (SD 27). Using their ending 3-day FBG average, 43% (28/65) of participants in this subset were now at goal (FBG<152 mg/dL, estimated HbA1c<7%), and 19% (12/65) met a much more aggressive goal for glycemic control (FBG<130 mg/dL, estimated HbA1c<6.5%).

Relationships between improvements in glycemic control and use of key nutritional-CBT features (therapy lessons, skills, and weekly goal setting) and self¬≠ reported diet and exercise behaviors were explored. Counts were summed for each participant and changes in FBG were examined for thirds of the entire population, from lowest to highest (tertiles) of feature or behavior type. In regression models, baseline average FBG, years since diagnosis of diabetes, and the duration of observation (the length of time between first and last value reported) were controlled for.

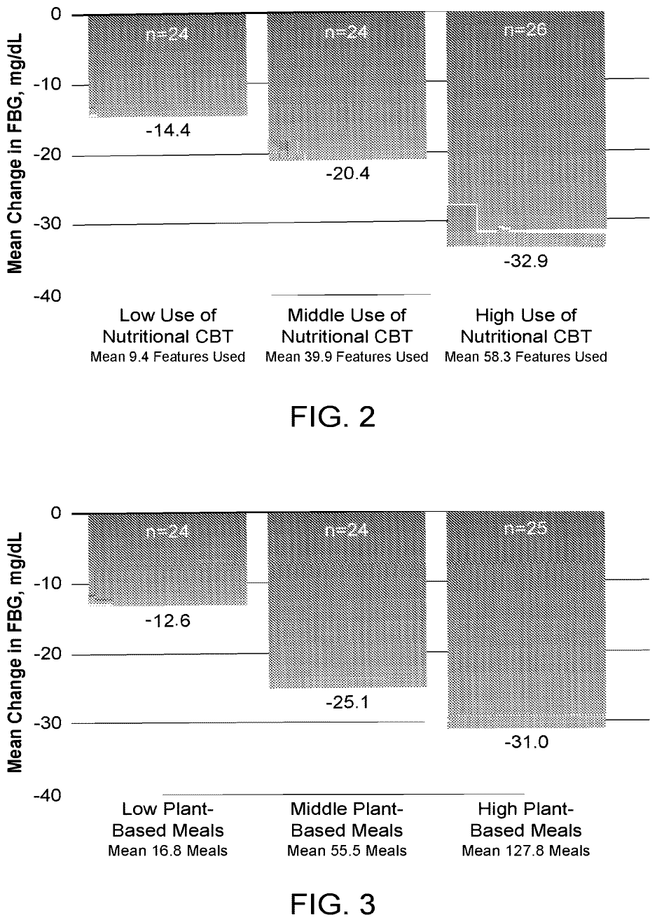

Of all variables explored, the use of nutritional-CBT content and the number of plant-based meals tracked demonstrated the strongest dose responses with improvements in FBG (FIGS. 2 and 3). In addition, a significant positive correlation was found between the use of the nutritional-CBT and change in dietary pattern (p<0.05 using linear regression).

In FIG. 2, the mean sum of nutritional-CBT actions completed for tertiles low to high were 9.4, 29.9 and 58.3. Pairwise comparison of the least square means controlling for baseline FBG, years since diagnosis and duration of FBG tracking was used to compare FBG across tertiles: low vs high tertile p=0.06, middle vs high tertile p=0.04.

In FIG. 3, the mean sum of plant-based meals reported for tertiles low to high were 17, 56 and 128. Pairwise comparison of the least square means controlling for baseline FBG, years since diagnosis and duration FBG tracked was used to compare FBG across tertiles: low vs high tertile p=0.18, middle vs high tertile p=0.46.

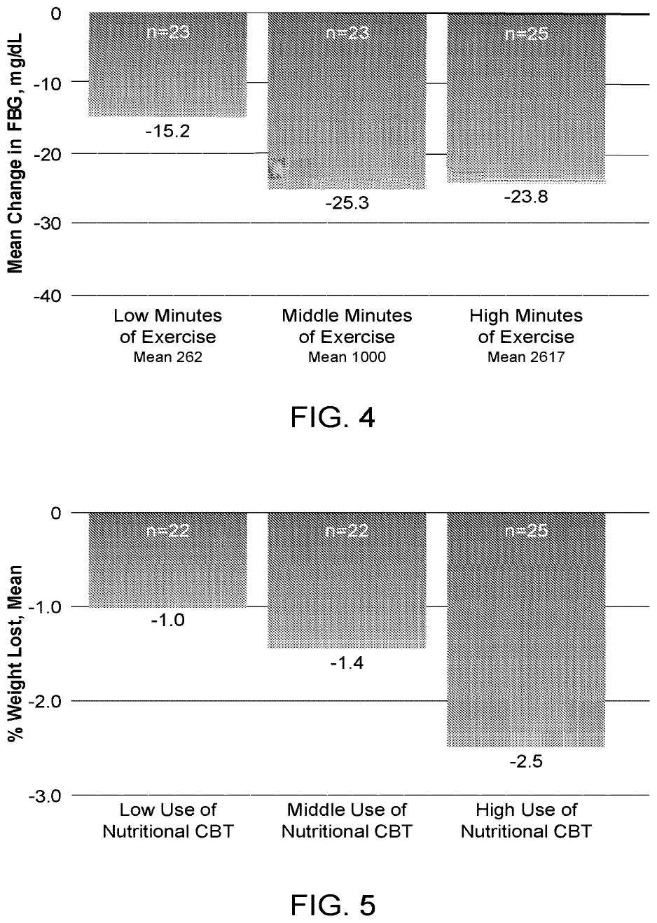

In the results, as seen for example in FIG. 4, it is possible to observe a trend between increased minutes of exercise reported and improvements in FBG. However, the dose-response pattern was not as clear as the pattern seen with nutritional-CBT and changes in diet.

FIG. 4 shows changes in FBG by tertile of minutes of exercise. In the bar chart of FIG. 4, mean minutes of total exercise over the 90 days reported for tertiles low to high were 262, 1,000 and 2,617. Pairwise comparison of the least square means controlling for baseline FBG, minutes of exercise in week 1, years since diagnosis and duration FBG tracked was used to compare FBG across tertiles: low vs high tertile p=0.08, middle vs high tertile p=0.48.

Weight loss also can lead to large improvements in glycemic control if participants lose 5-7% of body weight. The study explored whether changes in weight were associated with improvements in glycemic control. An average weight loss of 1.7% (SD 2.2) over a mean of 61 days was found between 3-day averages for baseline and last weight values reported. In a linear regression model, percent weight loss was significantly correlated with improvements in FBG (p=0.01). FIG. 5 shows a tertile analysis of nutritional-CBT use and changes in weight. The bar chart indicates that higher use may be associated with larger improvements in weight. There appeared to be greater weight loss in patients who used nutritional-CBT more extensively, but the degree of weight loss observed suggests that weight loss may not have been the major driver of glycemic changes.

FIG. 5 shows percent weight loss by tertile of nutritional CBT use. In FIG. 5, pairwise comparison of the least square means controlling for baseline FBG, years since diagnosis and duration FBG tracked was used to compare FBG across tertiles: low vs high tertile p=0.02, middle vs high tertile p=0.09.

In the study, usage data were examined to understand patterns of engagement, including frequency of use, time spent in each session of use, and duration of engagement. On average, participants used the app 4.5 days a week and spent 5 minutes in the app each day they used it. The duration of engagement was determined by the interval from the first day of use to the last day a participant either completed a nutritional-CBT task or entered a biometric value. 72% of the participants engaged with the program for 10 weeks or longer.

Participants were sent an online survey 10 weeks after starting their use of the app. 51% (38/74) completed the survey. The survey included questions about their experience using the app and asked about whether they would recommend the program to a friend or family member with diabetes. This question is referred to as a standardized Net Promoter Score (NPS) question. A calculated NPS score of Oto 30 is considered ‘good’, 30 to 70 is ‘great’ and over 70 is ‘excellent’. The average score for the healthcare industry in a recent report was 27. The calculated NPS score here was 58, which is “great”.

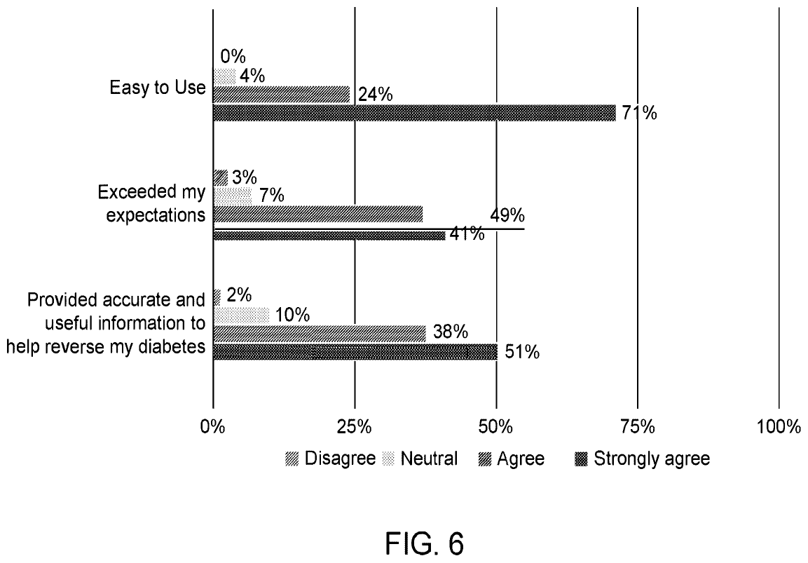

FIG. 6 indicates that overall participant opinions were positive in response to questions about ease of use, relevance of content, and ability to meet or exceed expectations.

The just-discussed study regarding a digital therapeutic delivering Nutritional—CBT resulted in clinically meaningful improvement in glycemic control. The mean decrease in FBG of −22.9 mg/d L corresponds to approximately a 1% reduction in HbA1c. An HbA1c reduction of 1% has been associated with a 21% decrease in diabetes related mortality and a 40% reduction in microvascular complications in the UK Prospective Diabetes Study with long-term follow up. This data is evidence that a software-only digital therapeutic in accordance with aspects of the present invention can serve as a standalone treatment.

The study results indicate a significant dose response between the degree of engagement in nutritional-CBT and improvements in glycemic control among adults with type 2 diabetes. This result is encouraging because it indicates that digitally¬≠ delivered behavioral therapy using only software has the potential to treat disease at scale.

Reductions in blood glucose were more significant and occurred faster than expected. Software-based treatment in accordance with aspects of the present invention can enable patients to make behavioral changes at a gradual pace. But the study results revealed more rapid blood sugar control than expected, with 38% of participants achieving a fasting blood glucose level less than 152 mg/dL (corresponding to an HbA1c<7%, which is commonly regarded as the goal for HbA1c for most patients with type 2 diabetes) and 16% achieving a fasting blood glucose less than 130 mg/dL (corresponding, on average to an HbA1c<6.5%, a much more aggressive goal for HbA1c) after an average of 69 days.

The foregoing results indicate that use of the software-based treatment in accordance with aspects of the invention can result in even greater improvements.

Improvements in blood glucose occurred in participants from across the country and with long standing diabetes. One hypothesis has been that only newly diagnosed patients will benefit from behavioral therapy. However, the study results indicate a strong efficacy signal in patients who were on average diagnosed with diabetes more than 10 years ago. The study also had excellent geographic diversity with participants from 32 states, including those with increasing prevalence of diabetes (e.g., Florida, Indiana and North Carolina).

According to the Centers for Disease Control (CDC), older individuals, especially those with diabetes and heart disease, are at higher risk of serious complications from COVID-19. Validated digital therapeutics can play an important role by being rapidly deployed into large populations to improve health, remotely monitor disease biometrics and self-reported symptoms, and escalate care as needed for triage and medical direction. This may be an effective way to protect the most vulnerable high-risk patients and keep them out of hospitals, emergency rooms and primary care clinics, alleviating burden on the system and associated costs.

When healthcare resources become severely stressed, and when healthcare resources simply are not readily available onsite to patients in remote areas, software¬≠ based treatment in accordance with aspects of the present invention can support and improve the health of patients with diabetes and other cardiometabolic conditions. This work highlights the potential for using digital therapeutics, once validated, to advance the care of these patients without increasing demand on the health system.

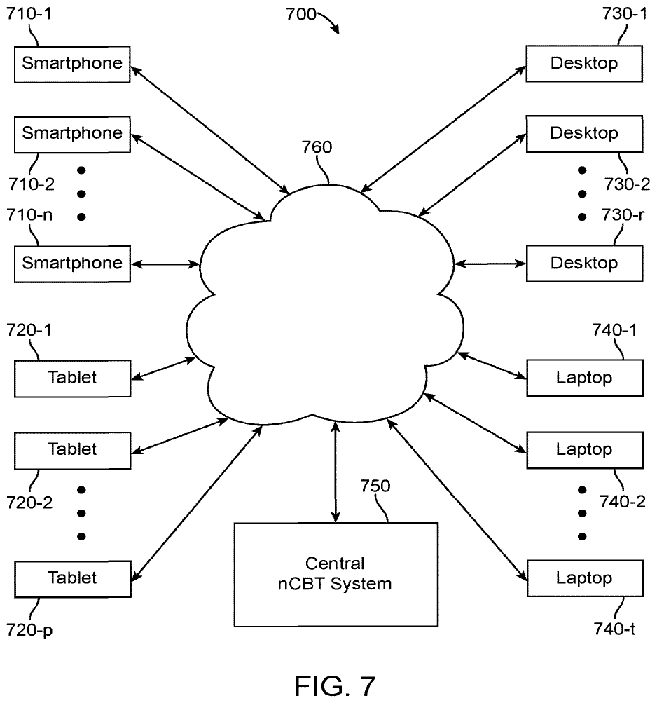

FIG. 7 shows, at a high level, aspects of a nutritional cognitive behavior therapy (nCBT) system 700 according to an embodiment, to illustrate aspects of system operation. A plurality of smartphones 710-1, . . . 710-n, tablets 720-1, . . . 720-p, desktops 730-1, . . . 730-r, and laptops 740-1, . . . , 740-t may communicate with a central nCBT system 750 to receive, provide, or exchange information as previously described. The smartphones 710-1, . . . 710-n, tablets 720-1, . . . 720-p, desktops 730-1, . . . 730-r, and laptops 740-1, . . . , 740-t may communicate with central nCBT system 750 either directly or through a network or cloud 760.

Depending on the embodiment, subjects may use a respective one (or, in some instances, more than one) of the smartphones 710-1, . . . 710-n, tablets 720-1, . . . 720-p, desktops 730-1, . . . 730-r, and laptops 740-1, . . . , 740-t to download a digital therapeutic either directly from central nCBT system 750, or through a service in or connected to the network or cloud 760. Subjects also may use a respective one or more of smartphones 710-1, . . . 710-n, tablets 720-1, . . . 720-p, desktops 730-1, . . . 730-r, and laptops 740-1, . . . , 740-t to receive therapy lessons and/or skill-building exercises either one at a time, or more than one at a time, from central nCBT system 750. Subjects may provide responses, biometric information, and the like to central nCBT system 750 through respective ones of smartphones 710-1, . . . 710-n, tablets 720-1, . . . 720-p, desktops 730-1, . . . 730-r, and laptops 740-1, . . . , 740-t. Central nCBT system 750 may in turn may generate one or more personalized notifications, including but not limited to guidance, progress reporting, treatment score, reminders, nudges, and rewards, to subjects who receive these via respective ones of smartphones 710-1, . . . 710-n, tablets 720-1, . . . 720-P, desktops 730-1, . . . 730-r, and laptops 740-1, . . . , 740-t. In an embodiment, responsive to biometric data from subjects, central nCBT system 750 may provide biometric notifications, including indications of danger levels.

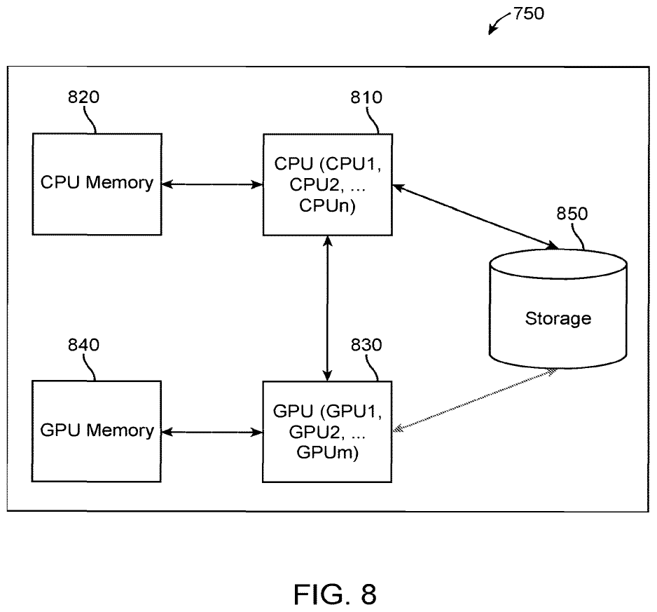

FIG. 8 shows aspects of the central nCBT system 750 according to an embodiment. It should be noted that, depending on the embodiment, there may be multiple instances of central nCBT system 750 distributed in different locations, all communicating with each other via the cloud 760 and, in some instances, communicating with different subjects via respective ones of smartphones 710-1, . . . 710-n, tablets 720-1, . . . 720-p, desktops 730-1, . . . 730-r, and laptops 740-1, . . . , 740-t. In an embodiment, a subject who is traveling may access a nearest or most convenient one of the multiple instances of central nCBT system 750. In such a configuration, data particular to the user is stored either centrally for the various central nCBT systems 750 to access, or locally in each of the various central nCBT systems 750. In the case of such local storage, updated information for respective subjects may be provided periodically to each of the various central nCBT systems 750.

An exemplary central nCBT system 750 may include one or more central processing units (CPUs) 810, each associated with CPU memory 820. Depending on the embodiment, each CPU 810 may have its own associated CPU memory 820. Alternatively, the CPUs 810 may share the CPU memory 820. Depending on the embodiment, one or more of the CPUs 810 may communicate with each other over a bus (not shown), to which CPU memory 820 also may be connected. In embodiments, CPU memory 820 may include volatile and/or non-volatile memory, and in some instances, non-transitory storage.

An exemplary central CnBT system 750 also may include one or more graphics processing units (GPUs) 830, each associated with GPU memory 840. Depending on the embodiment, each GPU 830 may have its own associated GPU memory 840. Alternatively, the GPUs 830 may share the GPU memory 840. Depending on the embodiment, one or more of the GPUs 830 may communicate with each other either directly or over a bus (not shown), to which GPU memory 840 also may be connected. In embodiments, GPU memory 840 may include volatile and/or non-volatile memory, and in some instances, non-transitory storage. Depending on the embodiment, one or more GPUs 830 may communicate with one or more CPUs 810 either directly over over a bus (not shown).

Storage 850 may take different forms, from one or more hard disk drives (HDD) to one or more solid state drives (SSD), to combinations of one or more HDD and one or more SSD.

Depending on the embodiment, one or more of the machine learning algorithms discussed above may reside in one or more GPUs 830, depending on the algorithm and its associated hardware requirements.

### Example 2: Pivotal Clinical Trial

Particularly relevant aspects of the trial discussed in this Example included the use of a nationally representative, diverse patient population (Table 4); use of investigators that mirror real-world prescribers; and robust study design employed to minimize bias and set a high comparison bar. For example, the control arm used in the study is Standard of Car (SOC) (i.e., gold standard care), not just treatment per usual; medication use and adjustment by investigators was not limited, only prandial insulin was excluded; and patients were not mandated nor incentivized to use the disclosed digital therapeutic, instead they were free to self-select dose.

TABLE 4
Patient population

| | | Standard | Digital |
|---|---|---|---|
| | | of Care | Therapeutic |
| Parameter/Category | Statistic | (n = 343) | (n = 325) |
| Age (yrs) | mean | 58.1 | 58 |
| % Female | % | 56.3 | 56 |
| Race | % | | |
| White | | 61.2 | 62.2 |
| Black or African American | | 29.2 | 29.5 |
| Asian | | 5.2 | 5.2 |
| American Indian or Alaskan | | 1.7 | 1.2 |
| Native | | | |
| Native Hawaiian or other | | 0.6 | 0.3 |
| Pacific Islander | | | |
| Ethnicity - Hispanic or | % | 14 | 17.2 |
| Latino | | | |
| Median Household income | mean | $67,737 | $69,789 |
| by ZIP code | | | |
| % High school degree or | % | 42% | 38.2 |
| some college but no degree | | | |

As seen at Table 4, the study included a nationally representative, diverse patient population. The population was recruited from 6 states, and included groups underrepresented in clinical trials, with historically poor access to care.

Participants had long-standing type 2 diabetes, high cardiovascular risk, multiple comorbidities, and extensive medication use (Table 5).

TABLE 5
Patient population medical details

| | | Standard | Digital |
|---|---|---|---|
| | | of Care | Therapeutic |
| Parameter/Category | Statistic | (n = 343) | (n = 326) |
| BMI (kg/m²) | mean | 34.7 | 34.6 |
| Baseline HbA1c (%) | mean | 8.1 | 8.2 |
| Years since diagnosis | mean | 10.9 | 11.0 |
| % on 2 or more | % | 67.5 | 68.4 |
| Antihyperglycemic | | | |
| medications | | | |
| Using antihypertensive | % | 71.7 | 67.1 |
| medications | | | |
| % on 2 or more number of | % | 66.5 | 69.0 |
| antihypertensive | | | |
| medications (for those | | | |
| treated for hypertensions, | | | |
| 67%% of participants) | | | |
| 10 year CV Risk score | mean | 15.1 | 15.1 |
| Number of Comorbidities | mean | 2.7 | 2.8 |

Baseline diabetes medications reveal robust background therapy compared with general diabetes population (Table 6).

TABLE 6
Background therapies

| | General Diabetes | Standard | Digital |
|---|---|---|---|
| Medication Class | Population (2018)¹ | or care | Therapeutic |
| All | 82.7% | 96.5% | 96.0% |
| Metformin | 59.5% | 79.9% | 80.1% |
| Sulfonylureas | 24.4% | 34.4% | 36.2% |
| SGLT2 inhibitors | <7.1% | 24.8% | 21.8% |
| GLP-1 analogues | <7.1% | 24.8% | 19.0% |
| Insulin | 25.6% | 19.5% | 17.8% |
| DPP-4 inhibitors | 10.8% | 13.7% | 17.2% |
| Thiazolidinediones | 3.3% | 5.0% | 5.5% |
| Meglitinides | — | 0.3% | 0.9% |

¹Fang et al. Trends in Diabetes Treatment and Control in U.S. Adults, 1999-2018. N Engl J Med 2021: 384; 2219-2228

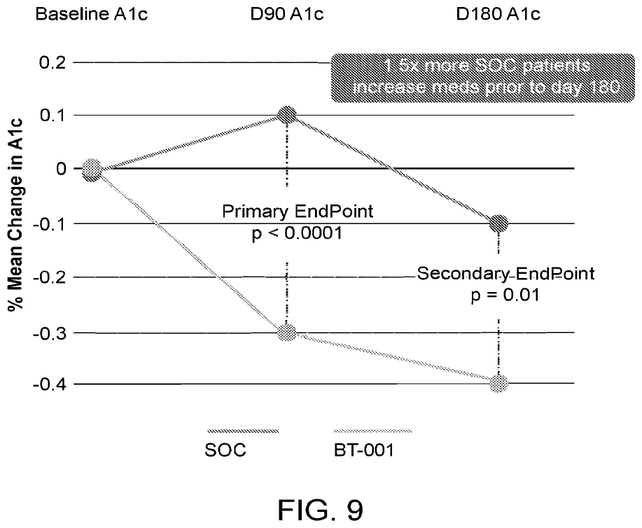

As shown in FIG. 9, the disclosed digital therapeutic (BT-001) demonstrated sustained and improved response at 180 days, with absolute Ale reduction advancing from 0.3% to 0.4%. The disclosed digital therapeutic reduced Ale despite on-study addition of more diabetes medication in the SOC control group. Notably, both primary (Ale between group delta—0.4%, p<0001) and secondary endpoints (Ale delta—0.3%, p.01) were met. Half of patients in the test arm achieved clinically meaningful changes with absolute mean Ale reduction of 1.3% (SD 0.8%) in this subgroup. The study showed robust safety data, with significantly fewer adverse events in the test arm (p<0.001). Digital therapeutic use was associated with multiple additional cardiometabolic benefits and lower medication and lower healthcare utilization.

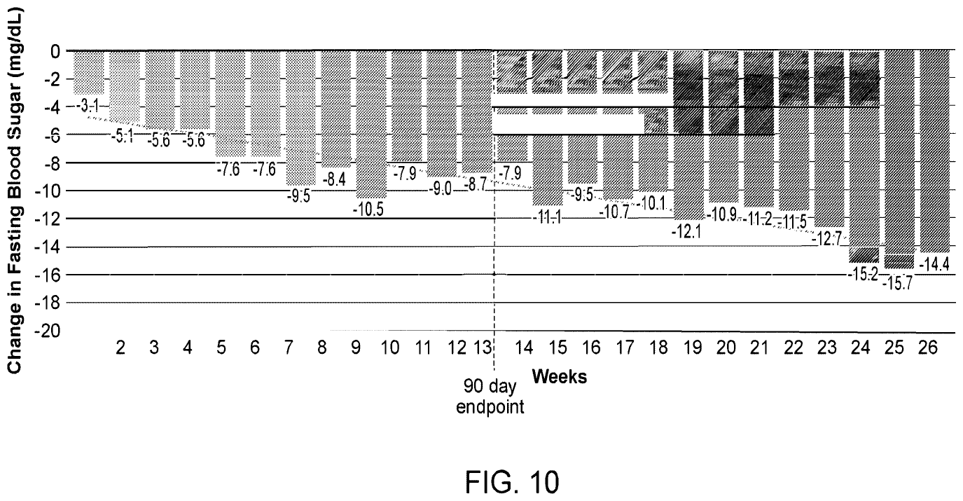

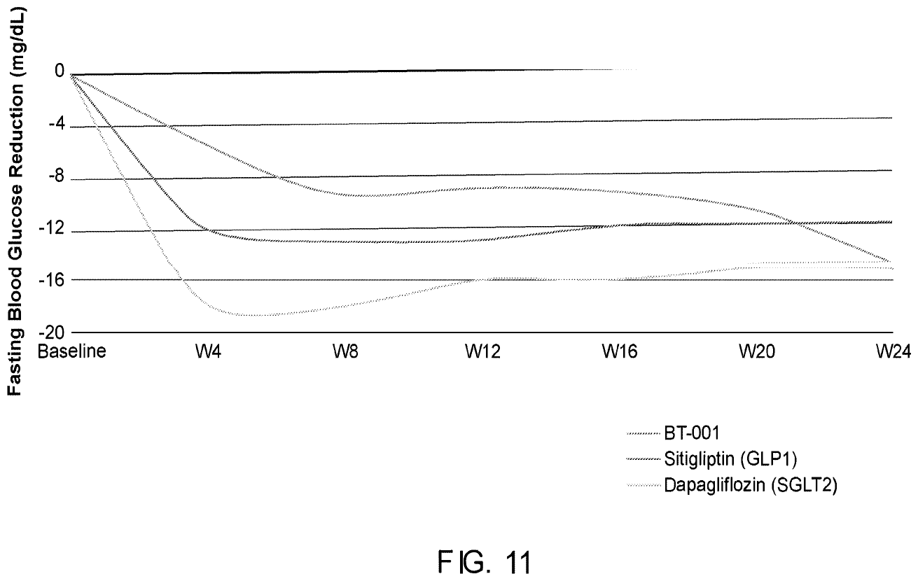

The trending average change in fasting blood glucose shows gradual and steady improvements, with no clear peak (FIG. 10). Shown at FIG. 11 is a graph illustrating trends in fasting blood glucose in different therapies (BT-001), Sitigliptin (GLP1), or Dapagliflozin (SGLT2) (see Ferrannini et al., Diabetes Care. (2010); 33: 2217-2224); Goldstein et al., Diabetes Care. (2007); 30(8): 1979-1987). It is to be understood that the data at FIG. 11 is from different studies with different trial designs and patient populations.

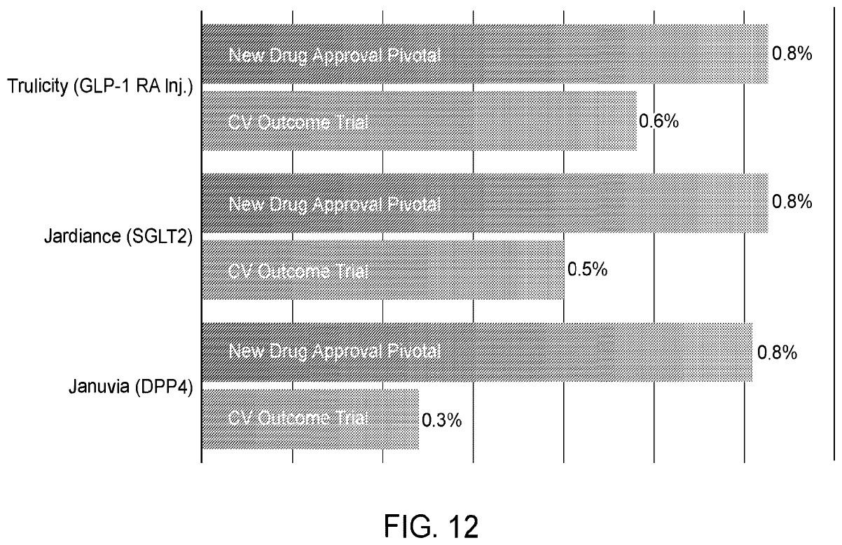

Turning to FIG. 12, depicted is a graph illustrating that cardiovascular outcome trials (CVOTs) show lower relative Ale reduction compared with new drug pivotal for same drug (Gerstein et al., Lancet. (2019); 394(10193): 121-130; Umpierrez et al., Diabetes Care. (2014); 37(8): 2168-2176; Zinman et al., New England J of Medicine. (2015); 373(22): 2117-2128; Roden et al., Lancet Diabetes Endocrinol. (2013); 1(3): 208-219; Green et al., The New England J of Medicine. (2015); 373(3): 232-242; Aschner et al., Diabetes Care. (2006); 29(12): 2632-2637). Hence, trial design may influence Ale reduction observed. The disclosed digital therapy pivotal trial design is more similar to diabetes cardiovascular outcome trials (Table 7).

TABLE 7
Digital therapy trial design comparison

| | | | Digital |
|---|---|---|---|
| | | | Therapeutic |
| Trial | New drug | CV outcome | pivotal |
| Characteristic | pivotal | trial | (BT-001) |
| Poorly controlled | Yes | Yes | Yes |
| diabetes at baseline | | | |
| Diabetes disease | <10 years | >10 years | >10 years |
| duration (mean) | | | |
| Baseline Therapy | Highly | Moderately | Minimally |
| Limited | (e.g., | (e.g., max of | (Only prandial |
| | Metformin | 2 drugs at | insulin |
| | monotherapy) | baseline) | excluded) |
| Comparison Arm | Placebo or | Standard of | Standard of |
| | singl eagent | care | care |
| Dosage of | Yes | Yes | No |
| Investigational | | | |
| Therapy Controlled | | | |

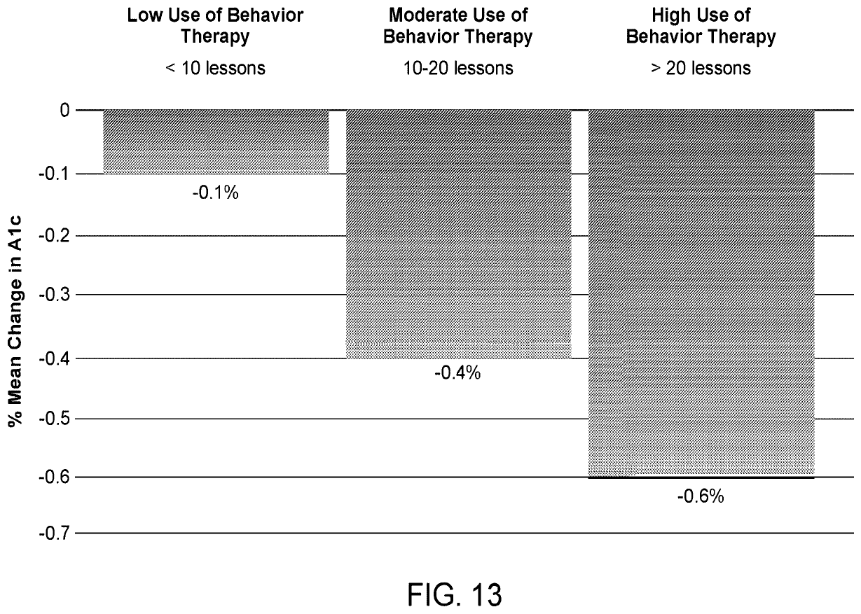

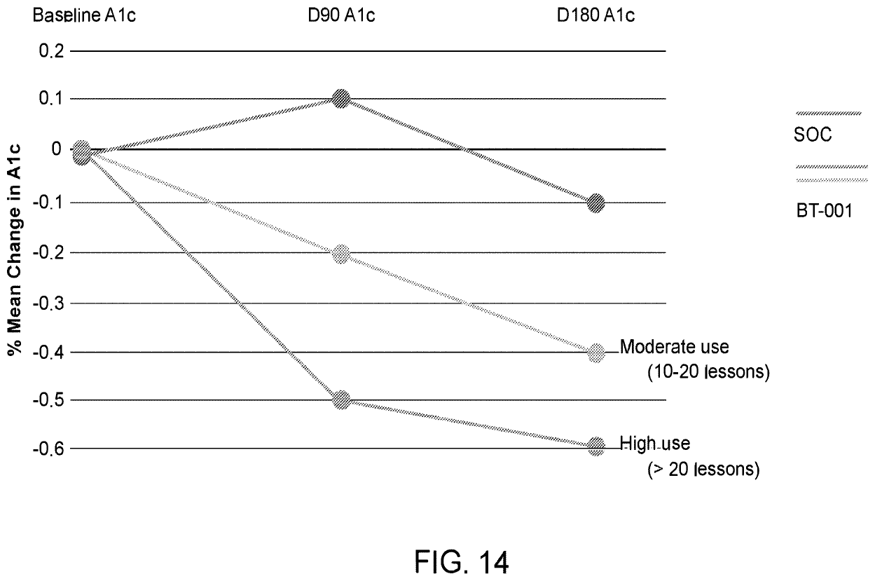

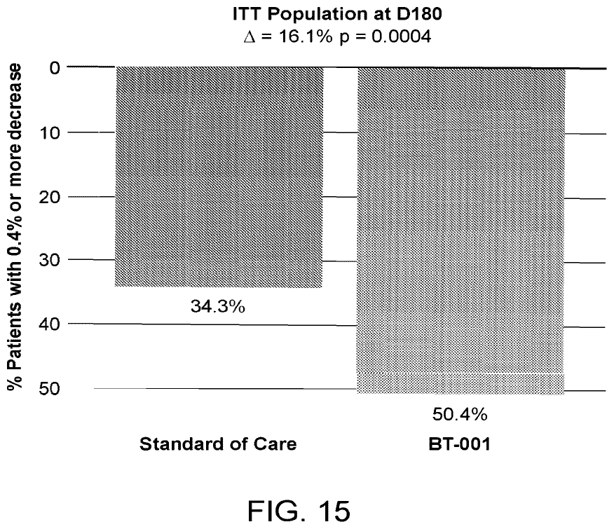

Patients were instructed to self-select dose of nCBT. Higher dose of nCBT lessons completed was associated with larger Ale improvements at 180 days (FIG. 13). The higher dose subgroup(>20 lessons) showed substantially greater Ale improvement compared to SOC control group (FIG. 14). 1.Sx more digital therapy patients achieved meaningful Ale change. Significant improvements were observed in the test group despite the use of fewer diabetes medications (FIG. 15). Notably, 30% of patients achieved a 1% or more Ale reduction vs. 17% for control group, p=0.001), and 30% of patients achieved blood sugar control target of Ale<7% vs. 20% SOC, p=0.009)

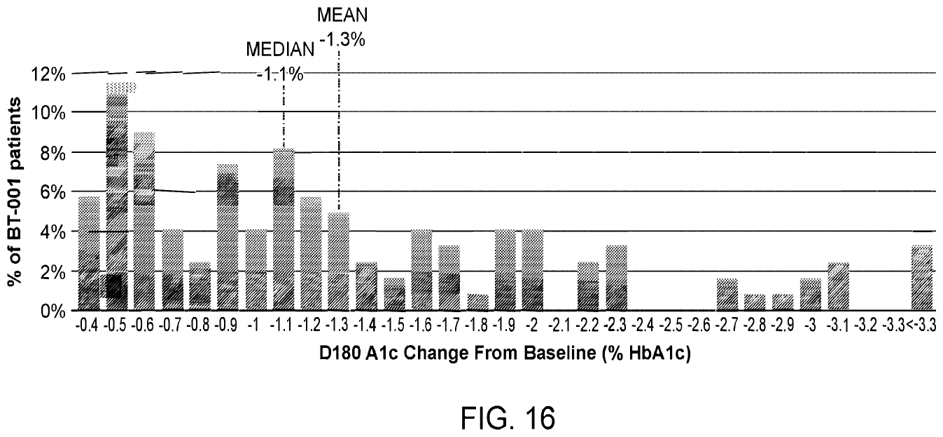

In the study, meaningful responders, defined as 0.4% or more Ale improvement, show a range of large improvements at 180 days (FIG. 16). Furthermore, 180 day safety data indicated significantly fewer adverse events (AEs) and fewer serious AEs (SAEs) (Table 8).

TABLE 8
180 day safety data

| | SOC (n = 343) | | | BT-001 (n = 325) | | |
|---|---|---|---|---|---|---|
| # subjects who | Subjects | | Events | Subjects | | Events |
| experienced | n (%) | | n | n (%) | | n |
| AE | 188 | (54.8%) | 324 | 135 | (41.5%) | 265 (p < |
| | | | | | | 0.001) |
| Serious AE | 24 | (7.0%) | 26 | 9 | (2.8%) | 9 (p = |
| | | | | | | 0.01) |
| AE possibly or | 0 | (0%) | 0 | 3 | (0.9%) | 4 |
| probably related | | | | | | |
| to study | | | | | | |
| intervention | | | | | | |
| AE related to | 0 | (0%) | 0 | 0 | (0.0%) | 0 |
| medical software | | | | | | |

Patients subjected to the disclosed digital therapy avoided more serious AEs commonly found in type 2 diabetes (Table 9).

TABLE 9
Patients on digital therapy avoidance of serious AEs

| | SOC (n = 343) | | | BT-001 (n = 325) | |
|---|---|---|---|---|---|
| # patients who | Subjects | | Events | Subjects | Events |
| experienced | n (%) | | n | n (%) | n |
| SAE | 24 | (7.0%) | 26 | 9 (2.8%) | 9 |
| SAE possibly | 14 | (4.1%) | 14 | 5 (1.5%) | 5 |
| related to | | | | | |
| diabetes/ | | | | | |
| cardiometabolic | | | | | |
| Cardiovascular | 6 | (1.7%) | 6 | 2 (0.6%) | 2 |
| Respiratory | 2 | (0.6%) | 2 | 1 (0.3%) | 1 |
| Infectious | 6 | (1.7%) | 6 | 2 (0.6%) | 2 |
| Other SAEs | 12 | (3.5%) | 12 | 4 (1.5%) | 4 |
| Death | 1 | (0.3%) | 1 | 0 (0.0%) | 0 |

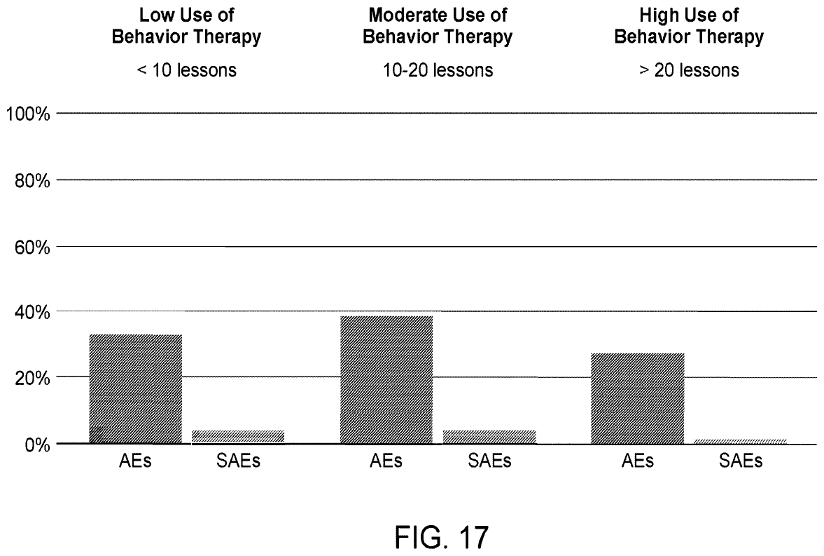

Advantageously, higher digital therapy dose was found to be associated with larger improvements, but not higher rates of AEs (FIG. 17).

Generally speaking, the safety profiles of top-performing diabetes drugs differ from the disclosed digital therapy (Table 10).

TABLE 10
Safety profiles

| Adverse Reaction | | | Digital Therapeutic |
|---|---|---|---|
| (>/=5%) | GLP1 | SGLT2 | (BT-001) |
| Nausea | Yes | No | No |
| Vomiting | Yes | No | No |
| Diarrhea | Yes | No | No |
| Abdominal Pain | Yes | No | No |
| Constipation | Yes | No | No |
| Female genital | No | Yes | No |
| mycotic infections | | | |
| Urinary track | No | Yes | No |
| infections | | | |
| Device related | N/A | N/A | <1% |
| adverse events | | | |

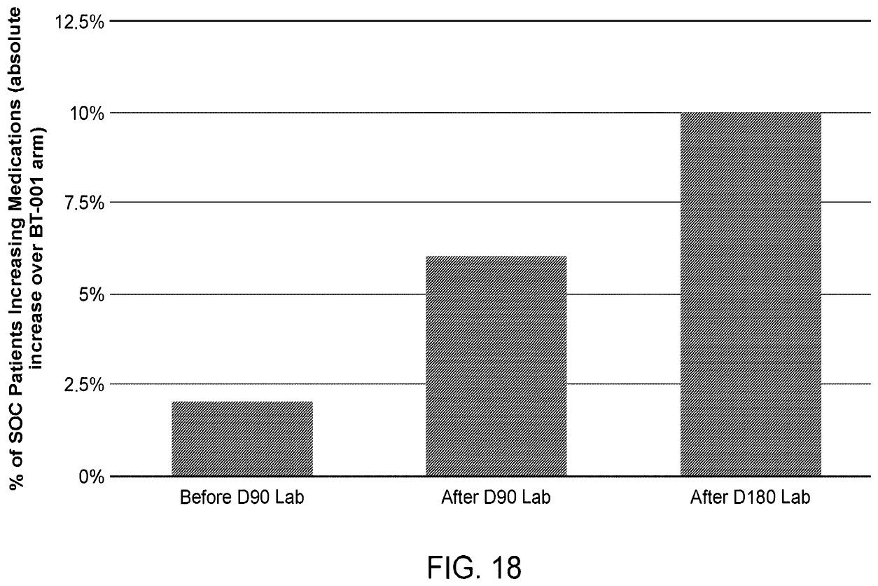

Antihyperglycemic medication utilization and healthcare utilization increased more in SOC control group with a widening gap over six months (FIG. 18). Patients relying on the disclosed digital therapy experienced fewer hospitalizations, ER visits, and outpatient visits over length of study.

During 180 days of use, patient engagement and persistence exceeded benchmarks for consumer and health wellness apps (Apptentive 2022 Mobile Customer Engagement Benchmark Report. % Retention at 90 days). Retention was found to be 94% in the group for which the disclosed digital therapy was tested. After 180 days, 81% of the patients were using the app, average minutes spent per day on the app was about 5.9 minutes, and the NPS score after 180 days was 61.

### Example 3: LivVita Study

Enrollment in a LivVita liver study has been completed. NAFLD/NASH affects over 64 million adults in the U.S., resulting in over $100 billion in direct healthcare costs annually. There are currently no FDA approved therapeutics for treating NASH/NAFLD.

The clinical study is evaluating the feasibility of nCBT to reduce liver fat and improve liver disease biomarkers as a potential treatment for fatty liver disease. This single arm interventional cohort study has completed enrollment of 22 adult patients from two specialized liver treatment clinics with data expected Q4 of 2022.

The primary object is to evaluate the feasibility and efficacy of nCBT in improving liver health in patients diagnosed NAFLD/NASH. The secondary objective is to gather user¬≠ experience feedback that will be used to improve usability of nCBT for future use in patients with NAFLD/NASH and evaluate intervention safety in this patient population. In addition, the study will explore the degree to which various non-invasive imaging technologies, composite scores, and serum laboratory biomarkers are sensitive to behavioral changes induces by nCBT.

Regarding changes in nCBT content for the LivVita study, no major changes were made to the product experience. Minimal changes were made to content knowing that additional changes can be made if needed prior to full a pilot or pivotal trial. No significant changes to skills or goal setting were made.

While aspects of the present invention have been described in detail above with reference to one or more embodiments, variations within the scope and spirit of the invention will be apparent to ordinarily skilled artisans. Accordingly, the invention should be considered as limited only by reference to the following claims.

1. A method for using machine learning for cognitive behavioral therapy, comprising:
providing, by at least one processor, a treatment plan for cognitive behavioral therapy in a mobile application to a user, the treatment plan for cognitive behavioral therapy comprising:
a series of lessons, wherein at least one lesson addresses one or more beliefs corresponding to one or more behaviors of the user; and
a plurality of exercises to provide an improvement corresponding to the one or more beliefs or to initiate a new behavior;
receiving, by the at least one processor, at least one of a plurality of responses or biometric data from the user corresponding with at least one lesson of the series of lessons or at least one exercise of the plurality of exercises;
identifying, by the at least one processor based on the at least one of the plurality of responses or the biometric data from the user, one or more endpoints for the user to achieve for a pre-defined duration of the treatment plan for cognitive behavioral therapy by applying at least one artificial intelligence (AI) model, and wherein the at least one Al model is updated using the plurality of responses of at least the user or biometric data from at least the user;
updating, by the at least one processor, the treatment plan for cognitive behavioral therapy based on one or more identified endpoints;
applying, by the at least one processor, the updated treatment plan for cognitive behavioral therapy to the user; and
wherein updating the treatment plan for cognitive behavioral therapy or identifying the one or more endpoints is further based on a target criterion and a progress criterion.

2. The method of claim 1, further comprising dynamically adjusting the one or more endpoints for the user between consecutive lessons or exercises, wherein the dynamic adjustment applies one or more machine learning algorithms trained using the plurality of responses and biometric data from a plurality of users.

3. The method of claim 1, further comprising modifying, for the user, a subsequent one of the lessons or at least one interactive exercise using the at least one processor, wherein the modifying applies one or more machine learning algorithms.

4. The method of claim 1, wherein the lesson is specific to a particular condition that the treatment plan for cognitive behavioral therapy is treating, or to understanding, addressing, or controlling particular human physiological attributes, or to understanding, addressing, or controlling particular human physiological responses, or developing certain desirable behaviors.

5. The method of claim 1, further comprising generating one or more personalized notifications and communicating the one or more personalized notifications to the user, the one or more personalized notifications selected from a group consisting of guidance, progress reporting, treatment score, reminders, nudges, and rewards.

6. The method of claim 1, wherein the identifying relies at least in part on performance by the user in reaching the one or more endpoints previously set.

7. The method of claim 1, wherein one or more of the series of lessons are interactive, and the user inputs information into the mobile application either voluntarily or as the mobile application provides a prompt.

8. The method of claim 1, further comprising providing biometric notifications via a protected data channel in response to entry of the biometric data of the user, optionally wherein the biometric notifications indicate danger levels.

9. The method of claim 1, the method further comprising determining one or more treatment changes or behavioral modifications for the user.

10. A system for machine learning for cognitive behavioral therapy, comprising:
one or more processors coupled with memory, configured to:
provide a treatment plan for cognitive behavioral therapy in a mobile application to a user, the treatment plan for cognitive behavioral therapy comprising:
a series of lessons, wherein at least one lesson addresses one or more beliefs corresponding to one or more behaviors of the user; and
a plurality of exercises to provide an improvement corresponding to the one or more beliefs or to initiate a new behavior;
receive at least one of a plurality of responses or biometric data from the user corresponding with at least one lesson of the series of lessons or at least one exercise of the plurality of exercises;
identify, based on the at least one of the plurality of responses or the biometric data from the user, one or more endpoints for the user to achieve for a pre-defined duration of the treatment plan for cognitive behavioral therapy by applying at least one artificial intelligence (AI) model, and wherein the at least one Al model is updated using the plurality of responses of at least the user or biometric data from at least the user;
update the treatment plan for cognitive behavioral therapy based on one or more identified endpoints;
apply the updated treatment plan for cognitive behavioral therapy to the user; and
wherein updating the treatment plan for cognitive behavioral therapy or identifying the one or more endpoints is further based on a target criterion and a progress criterion.

11. The system of claim 10, wherein the one or more processors are further configured to dynamically adjust the one or more endpoints for the user between consecutive lessons or exercises, wherein the dynamic adjustment applies one or more machine learning algorithms trained using the plurality of responses and biometric data from a plurality of users.

12. The system of claim 10, wherein the one or more processors are further configured to modify, for the user, a subsequent one of the lessons or at least one interactive exercise using the one or more processors, wherein the modifying applies one or more machine learning algorithms.

13. The system of claim 10, wherein the lesson is specific to a particular condition that the treatment plan for cognitive behavioral therapy is treating, or to understanding, addressing, or controlling particular human physiological attributes, or to understanding, addressing, or controlling particular human physiological responses, or developing certain desirable behaviors.

14. The system of claim 10, wherein the one or more processors are further configured to generate one or more personalized notifications and communicate the one or more personalized notifications to the user, the one or more personalized notifications selected from a group consisting of guidance, progress reporting, treatment score, reminders, nudges, and rewards.

15. The system of claim 10, wherein identifying relies at least in part on performance by the user in reaching the one or more endpoints previously set.

16. The system of claim 10, wherein one or more of the series of lessons are interactive, and the user inputs information into the mobile application either voluntarily or as the mobile application provides a prompt.

17. The system of claim 10, wherein the one or more processors are further configured to provide biometric notifications via a protected data channel in response to entry of the biometric data of the user, optionally wherein the biometric notifications indicate danger levels.

18. The system of claim 10, wherein the one or more processors are further configured to determine one or more treatment changes or behavioral modifications for the user.

19. A non-transitory computer readable medium (CRM) comprising one or more instructions stored thereon and executable by one or more processors to:
provide a treatment plan for cognitive behavioral therapy in a mobile application to a user, the treatment plan for cognitive behavioral therapy comprising:
a series of lessons, wherein at least one lesson addresses one or more beliefs corresponding to one or more behaviors of the user; and
a plurality of exercises to provide an improvement corresponding to the one or more beliefs or to initiate a new behavior;
receive at least one of a plurality of responses or biometric data from the user corresponding with at least one lesson of the series of lessons or at least one exercise of the plurality of exercises;
identify, based on the at least one of the plurality of responses or the biometric data from the user, one or more endpoints for the user to achieve for a pre-defined duration of the treatment plan for cognitive behavioral therapy by applying at least one artificial intelligence (AI) model, and wherein the at least one Al model is updated using the plurality of responses of at least the user or biometric data from at least the user;
update the treatment plan for cognitive behavioral therapy based on one or more identified endpoints;
apply the updated treatment plan for cognitive behavioral therapy to the user; and
wherein updating the treatment plan for cognitive behavioral therapy or identifying the one or more endpoints is further based on a target criterion and a progress criterion.

20. The CRM of claim 19, wherein the one or more instructions stored thereon and executable by the one or more processors are further configured to:
dynamically adjust the one or more endpoints for the user between consecutive lessons or exercises, wherein the dynamic adjustment applies one or more machine learning algorithms trained using the plurality of responses and biometric data from a plurality of users.

21. The CRM of claim 19, wherein the one or more instructions stored thereon and executable by the one or more processors are further configured to modify, for the user, a subsequent one of the lessons or at least one interactive exercise using the one or more processors, wherein the modifying applies one or more machine learning algorithms.

22. The CRM of claim 19, wherein the lesson is specific to a particular condition that the treatment plan for cognitive behavioral therapy is treating, or to understanding, addressing, or controlling particular human physiological attributes, or to understanding, addressing, or controlling particular human physiological responses, or developing certain desirable behaviors.

23. The CRM of claim 19, wherein the one or more instructions stored thereon and executable by the one or more processors are further configured to generate one or more personalized notifications and communicate the one or more personalized notifications to the user, the one or more personalized notifications selected from a group consisting of guidance, progress reporting, treatment score, reminders, nudges, and rewards.

24. The CRM of claim 19, wherein identifying relies at least in part on performance by the user in reaching the one or more endpoints previously set.

25. The CRM of claim 19, wherein one or more of the series of lessons are interactive, and the user inputs information into the mobile application either voluntarily or as the mobile application provides a prompt.

26. The CRM of claim 19, wherein the one or more instructions stored thereon and executable by the one or more processors are further configured to provide biometric notifications via a protected data channel in response to entry of the biometric data of the user, optionally wherein the biometric notifications indicate danger levels.

27. The CRM of claim 19, wherein the one or more instructions stored thereon and executable by the one or more processors are further configured to determine one or more treatment changes or behavioral modifications for the user.

28. The method of claim 1, wherein the one or more beliefs correspond to a health condition comprising a cardiometabolic disorder selected from the group consisting of: type 2 diabetes, gestational diabetes, hypertension, obesity, dyslipidemia, hyperlipidemia, hypertriglyceridemia, non-alcoholic fatty liver disease, non-alcoholic steatohepatitis, hypercholesterolemia, familial hypercholesterolemia, heart disease, coronary artery disease, and chronic kidney disease.

29. The method of claim 1, wherein the one or more beliefs are selected from the group consisting of: a belief regarding an ability of the user to update or control the one or more behaviors of the user, a belief regarding nutrients, and a belief regarding eating or exercising.

30. The method of claim 1, wherein the one or more beliefs comprise one or more maladaptive beliefs corresponding to one or more dietary and/or lifestyle behaviors of the user.

31. The method of claim 30, wherein the one or more maladaptive beliefs are selected from the group consisting of: ability of the user to change and/or control behaviors; beliefs of the user regarding nutrients, optionally including macronutrients, and importance of various food types; and beliefs of the user regarding experiences in eating and/or exercising.

32. The method of claim 1, wherein the series of lessons comprises one or more topics comprising exploring beliefs, Type 2 Diabetes, blood sugar, protein, affordability, exercise, hunger, weight, comfort food, control, loyalty, ability to change, healing, power of beliefs, stress, response to stress, sleep, connection, opportunity, meaning, purpose, strength/resistance exercise, caring for ourselves, empowerment, craving, evolving, or any combination thereof.

33. The method of claim 32, wherein the series of lessons comprises two or more, three or more, four or more, five or more, ten or more, fifteen or more, twenty or more, twenty-five or more, or all of the topics.

34. The method of claim 1, wherein the at least one Al model is selected from the group consisting of: Linear Regression, Logistic Regression, Linear Discriminant Analysis, Classification and Regression Trees, Decision Trees, Clustering, Inductive Logic Processing, Stochastic Modeling, Statistical Modeling, Case-Based Reasoning, Bayes and Naive Bayes, K-Nearest Neighbors (KNN), Learning Vector Quantization (LVQ), Support Vector Machines (SVM), Random Forest, Boosting, AdaBoost, and an artificial neural network selected from the group consisting of convolutional neural networks (CNN), recurrent neural networks (RNN), and recurrent convolutional neural networks (RCNN).

35. The method of claim 7, wherein the information is selected from the group consisting of: audio recordings, video recordings, photographs, journal entries, and other written text.

36. The method of claim 1, further comprising interacting with the user so that the user either accepts the one or more endpoints or identifies other endpoints to be achieved.

37. The method of claim 1, wherein the one or more endpoints comprise one or more of diet, exercise, and medication.

38. The method of claim 1, wherein the new behavior comprises a new dietary behavior, a new lifestyle behavior, or any combination thereof.

39. The method of claim 1, further comprising modifying, for the user, a subsequent one of the lessons or at least one interactive exercise using the at least one processor, wherein the modifying applies one or more machine learning algorithms trained using responses and biometric data from a plurality of users.

40. The method of claim 1, further comprising dynamically adjusting the one or more endpoints for the user between consecutive lessons or exercises based on responses and biometric data from a plurality of users.
